(19) United States
(12) Patent Application Publication
(10) Pub. No.: US 2014/0366884 A1
(43) Pub. Date: Dec. 18, 2014

(54) PATIENT INTERFACE DEVICE FIXABLE TO THE PATIENT VIA A BONE ANCHOR INSIDE THE PATIENT'S MOUTH

(71) Applicant: KONINKLIJKE PHILIPS N.V., EINDHOVEN (NL)

(72) Inventors: Fransciscus Joannes Leonardus Everaerts, Weert (NL); Sander Theodoor Pastoor, Utrecht (NL); Steven Ernest Franklin, Eindhoven (NL)

(21) Appl. No.: 14/362,416

(22) PCT Filed: Dec. 11, 2012
(86) PCT No.: PCT/IB2012/057159

§ 371 Date: Jun. 3, 2014

Related U.S. Application Data
(60) Provisional application No. 61/569,334, filed on Dec. 12, 2011.

(51) Int. Cl. A61M 16/06

(52) U.S. Cl. CPC A61M 16/0605
USPC 128/206.21

(57) ABSTRACT

The present invention relates to a patient interface device and system for delivering continuous airway pressure to a patient (14). The patient interface system comprises a patient interface (12) and at least one bone anchor (24, 26) fixed to the patient (14). The patient interface (12) is attached to the patient (14) by connection to the at least one bone anchor (24, 26). This is realized by coupling elements (32, 34) as part of the patient interface device.

### FIELD OF THE INVENTION

The present invention relates to a patient interface device and a patient interface system for delivering a gas to a patient. Such patient interfaces are often referred to as facial masks or respiratory interface devices. The present invention relates further to a method for attaching a patient interface device to a patient and the use of a bone anchor for connecting a patient interface device to it.

### BACKGROUND OF THE INVENTION

For several chronic disorders and diseases, e.g. Obstructive Sleep Apnea (OSA), a long-term attachment of a patient interface to a patient is necessary or at least advisable. Therein, airway pressure is often used. For example, sleep apnea can be treated in an extremely effective way by using airway pressure (e.g. CPAP, BiPAP). Therefore, the afore-mentioned long-term use of the patient interface takes place during the sleeping time of the patient.

As an example for this application, four basic types of patient interfaces shall be mentioned:

nasal masks, which fit over the nose and deliver a gas through the nasal passages,

oral masks, which fit over the mouth and deliver a gas through the mouth,

full face masks, which fit over both, the nose and the mouth, and deliver a gas to both, and

nasal pillows, which are regarded as masks as well within the scope of this invention and which consist of small nasal inserts that deliver the a gas directly into the nasal passages.

If the term “mask” is used herein, this shall not be understood as a limitation to this special embodiment rather than referring to patient interfaces as described herein in general, where applicable and if not stated otherwise.

An example for a patient interface device designed as a full face mask for the usage with continuous airway pressure is described in U.S. Pat. No. 7,640,933 B1.

Resulting from the afore-mentioned long-term usage of those patient interfaces, an interface attachment can often be the reason for discomfort of the patient. This is especially disturbing for the patient if the patient interface is used, for example, for the mentioned treatment of sleep apnea. Especially at that time, i.e. in sleep, a patient wants to and should have no disturbing influences.

Commonly known masks all show a fixation to the patient by using some sort of headgear. Although the mask and headgear can vary in size, shape and material, the headgear normally covers significant parts of the whole head and is designed of relatively robust materials due to the use in a continuous airway pressure application.

### SUMMARY OF THE INVENTION

It is an object of the present invention to provide a patient interface device, which is attachable in such a way to a patient that it avoids the use of cumbersome straps and headgear, reducing the discomfort and inconvenience a patient feels by wearing the attached patient interface in a long-term application.

According to an aspect of the present invention, a patient interface device is provided, comprising:

a patient interface for delivering a gas to a patient, and

at least one coupling element arranged at said patient interface,

wherein said patient interface is attachable to the patient by connecting said at least one coupling element to at least one tissue anchor, said at least one tissue anchor being fixed to the patient.

According to another aspect of the present invention, a patient interface system is provided, comprising:

a patient interface device of the kind mentioned before and

at least one tissue anchor corresponding to said at least one coupling element and being fixable to tissue of the patient.

Contrary to the proposed patient interface system the proposed patient interface device does not comprise elements that are fixable or fixed to the patient.

According to another aspect of the present invention, a method for attaching a patient interface of a patient interface device of the kind mentioned before to a patient is provided, comprising:

arranging the patient interface at the patient and

connecting at least one coupling element of the patient interface system to a corresponding tissue anchor, said tissue anchor being fixed to the patient.

According to another aspect of the present invention, it is provided the use of a bone anchor for connecting a patient interface device of the kind mentioned before to it

Preferred embodiments of the invention are defined in the dependent claims. It shall be understood that the patient interface system may have the same or similar preferred embodiments as explained hereinafter and as defined in the dependent claims for the patient interface device. It shall be further understood that the claimed method has similar and/or identical preferred embodiments as the claimed device and as defined in the dependent claims.

Using a bone anchor as a way for attaching a mask or a patient interface in general to a patient instead of an, in general, large headgear provides the possibility to reduce the parts necessary for the fixation to a smaller area on the patient, e.g. on the patient's head. The result is a patient interface device, which avoids a large attaching arrangement, thereby reducing the discomfort and inconvenience for the patient during a long-term use of the patient interface. By comprising a coupling element, the patient interface may be attached quite easily to the anchor, since coupling element and patient interface can be moved independently from one another to a certain degree.

According to another embodiment of the device of the present invention, the at least one coupling element comprises a solid bar. A solid bar resembles a coupling element that allows a stiff and reliable fixation and attachment of the mask to the patient.

According to another embodiment of the device of the present invention, the at least one coupling element comprises a flexible element. The usage of a flexible element allows for an easier attachment of the patient interface to the patient. Further, it gives the patient again a more convenient and comfortable attachment of the patient interface. This is due to the possibility that the patient interface and its attachment to the patient may actually compensate small dislocations of the patient interface which may occur especially during sleep due to the turning of the patient.

According to another embodiment of the device of the present invention, the patient interface device comprises a plurality of coupling elements and in an alternate embodiment 2 or 4 coupling elements. The usage of at least two coupling elements allows for a uniform attachment of the patient interface to the patient due to the distribution of force towards different anchor positions. For this, each coupling element is preferably designed to be attached to one tissue anchor. However, embodiments wherein two or more coupling elements are attached to one anchor together lie also within the scope of the present invention.

According to another embodiment of the device of the present invention, the at least one coupling element is designed to be connected to a tissue anchor in the patient's upper jaw, or alternatively the patient's lower jaw. Having different positions of the tissue anchor, e.g. in the upper and/or lower jaw, specifically designed coupling elements for each anchor position give the optimum performance for the attachment of the patient interface to the patient. This design of the coupling elements is given by their respective arrangement site at the patient interface, their length and their movability, for example.

According to another embodiment of the system of the present invention, the at least one tissue anchor comprises an attachment post, an attachment plate, or a combination thereof. The usage of an attachment post allows for a simple rod or pin-like anchor, which is easy to arrange at the patient and which allows easy fixation. The usage of an attachment plate allows for a reliable and well positioned fixation of the patient interface. This is especially the case in a preferred arrangement wherein the attachment plate is attached to the patient by using multiple attachments for one such plate. This plate may serve as a kind of docking station where a larger end of a coupling element can be connected to. This may especially be helpful for attaching and detaching steps, since such an arrangement might be quite easy to access and to operate.

According to another embodiment of the system of the present invention, the at least one tissue anchor is adapted to be arranged inside the mouth of the patient. An arrangement of the anchor inside the mouth of the patient locates the anchor at a position where it is commonly not seen by someone else. This increases the comfort and convenience of the patient in the everyday life when the patient interface is not worn.

According to another embodiment of the system of the present invention, the at least one tissue anchor is designed as a bone anchor. Bone tissue has the advantage of being a relatively strong base for attaching an anchor to. Further, the knowledge of attaching other kinds of anchors to bone tissue known from surgery can be used and, therefore, a very strongly fixed anchor is achieved. Further, a bone anchor is permanently fixable to the patient. With such an arrangement, the patient avoids the necessity known from the large headgears to accurately position and fixate the patient interface to the head, since the attaching positions of the patient interface to the anchor are already and permanently present.

According to another embodiment of the system of the present invention, the at least one bone anchor is adapted to be arranged in the patient's upper jaw or, in an alternative embodiment of the present invention, in the patient's lower jaw. The attachment of the anchor into either the upper or the lower jaw results in a fixation of the mask to an easy accessible, though not easily noticeable area. Further, an opening of the mouth is necessary for the attachment of this patient interface to such an arranged anchor, which aids in the delivery of the continuously supplied pressurized air.

According to another embodiment of the system of the present invention, the tissue anchor is fixed to the patient by a screw, glue, cement, staples or a combination thereof. Attaching the anchor using these methods results in a strongly fixed anchor as a basis for an attachment of the patient interface to the patient. From these fixation methods, attaching the anchor using a screw is preferred.

In all mentioned embodiments of the present invention, the patient interface device is preferably designed to deliver pressurized gas to a patient. Generally, however, the proposed mask apparatus is adapted to deliver any kind of gas to a patient. Hence, whenever “air” or “a gas” is mentioned herein, this term shall not be understood as limited to pure “air”, but shall include any kind of air or gas that shall be delivered to a patient, e.g. air enriched in oxygen, a gas mixture comprising oxygen and a medicine.

Preferred embodiments of the invention are defined in the dependent claims.

### BRIEF DESCRIPTION OF THE DRAWINGS

These and other aspects of the invention will be apparent from and elucidated with reference to the embodiment described hereinafter. In the following drawings

FIG. 1 shows a schematic perspective view of a patient wearing a patient interface device according to the present invention,

FIG. 2 shows a schematic and partly sectional side view looking at the left side of the patient's head of FIG. 1,

FIG. 3 shows a schematic side view of an upper and a lower jaw of a patient,

FIG. 4 shows a schematic view of a part of a patient's lower jaw, and

FIG. 5 shows an enlarged schematic view of an anchor embodiment according to the present invention.

### DETAILED DESCRIPTION OF THE INVENTION

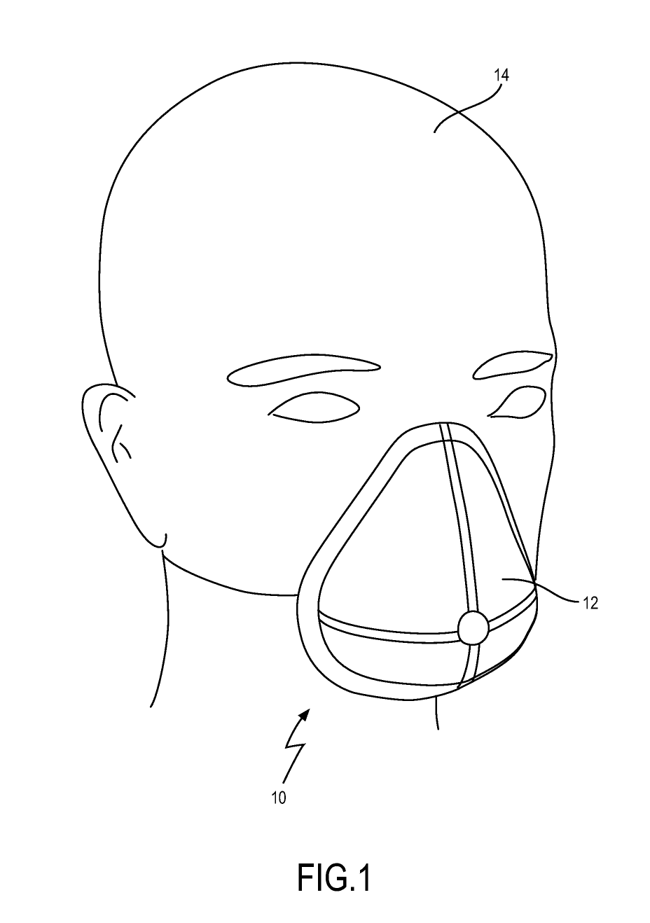

An embodiment of a patient interface device according to the present invention is shown and described throughout FIGS. 1 to 5 and designated by the reference numeral 10 in its entirety.

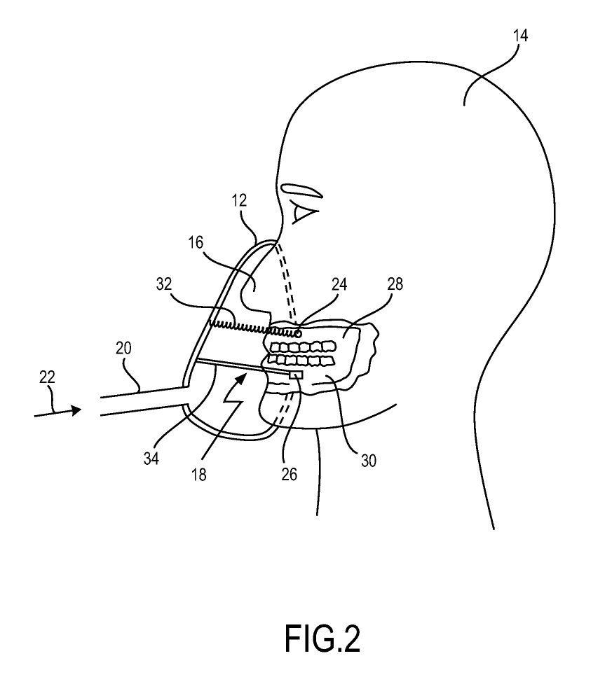

Said patient interface device 10 comprises a patient interface 12 which is attached to a patient's head 14. As can be seen especially in FIG. 2, this arrangement is in that way that the patient interface 12 covers the nose 16 as well as the mouth 18 of the patient's head 14 at the same time. In this particular embodiment, the patient interface 12 is designed as a mask. The patient interface device 10 further comprises a hose 20. This hose 20 is attached to the patient interface 12. Through the hose 20, pressurized gas is supplied from an outside source (not shown) via the patient interface 12 to the patient's nose 16 and mouth 18. This is indicated by an arrow 22. Although, pressurized gas is mentioned as the gas supplied to the patient 14 in this case, any other gas that shall be supplied to a patient may be used with the patient interface device 10 within the present invention.

In order to attach the patient interface 12 to the patient's head 14, bone anchors 24 and 26 are provided. Together, patient interface device 10 and bone anchors 24 and 26 are forming a patient interface system.

In this exemplary embodiment, the bone anchor 24 is arranged in the upper jaw 28. The bone anchor 26, on the other hand, is arranged in the lower jaw 30.

Although the description for the patient interface system is made in this exemplary embodiment for two bone anchors 24 and 26, it goes without mentioning that an attachment of the patient interface 12 to the patient's head 14 can also be realized by just one bone anchor 24 or 26. Also, it goes without mentioning that such a bone anchor 24 and/or 26, which is in this embodiment arranged on the left side of the respective patient's jaw 28 or 30, may also have another respective bone anchor on the right side of the respective jaw (not shown).

In order to achieve a fixation of the patient interface 12 to the patient's head 14, the patient interface device 10 further comprises coupling elements 32 and 34. In this embodiment, the coupling element 32 is designed as a flexible element. This flexible coupling element 32 connects the patient interface 12 with the bone anchor 24. The coupling element 34 is designed as a solid bar. This solid bar coupling element 34 connects the patient interface 12 to the bone anchor 26.

Although the description is made for a flexible and a solid bar coupling element used simultaneously in this exemplary embodiment, it goes without saying that an embodiment wherein only flexible elements or only solid bar coupling elements are used also lies within the scope of the present invention.

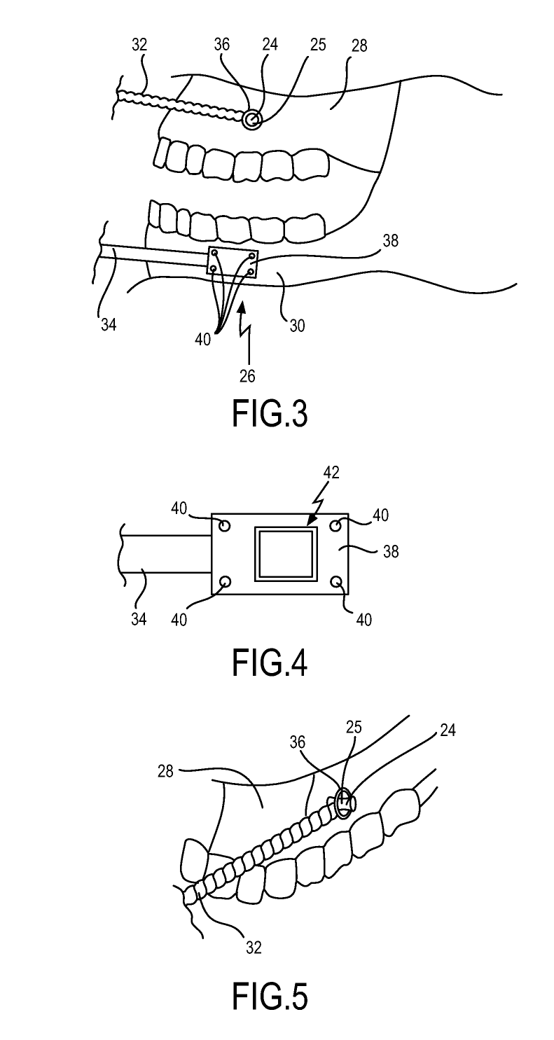

The embodiments of the bone anchors 24 and 26 are described in more detail in the enlarged view of the upper jaw 28 and lower jaw 30 of the head of the patient 14 in FIG. 3. As can be seen, in this embodiment bone anchor 24 is designed as a pin or a rod-like element and resembles an attachment post 25. Therefore, this attachment post 25, i.e. the bone anchor 24, is attached in a single point to the upper jaw 28. In order to attach the flexible coupling element 32 to the attachment post bone anchor 24, the coupling element 32 comprises a ring 36. This ring 36 can be hooked on the attachment post 25, i.e. the bone anchor 24.

The design of the flexible coupling element 32 can be for example an elastic band like a rubber band or a non-elastic, but also flexible strap.

The bone anchor 26 is in this embodiment designed as an attachment plate 38. This attachment plate 38 is fixed to the underlying bone of the lower jaw 30 via multiple attachment points. These attachment points can be, for example, screws 40. However, an attachment wherein glue, cement or staples are used in order to attach this attachment plate 38 to the bone tissue of the lower jaw 30 or also upper jaw 28, lies also within the scope of the present invention. It goes without mentioning that an attachment post like the bone anchor 24 can also be attached to an underlying bone of any of the two jaws 28 or 30 via the afore-mentioned fixation methods, i.e. glue, cement, staples or screws. Also, where possible, combinations of these fixation methods are possible.

As shown in more detail in FIG. 4, the connection of the coupling element 34 at the attachment plate 38 is realized via a click mechanism 42. However, this special embodiment is not meant to limit the scope of the present invention, and any other locking mechanism for attaching a solid coupling element like the coupling element 34 or also a flexible coupling element like the coupling element 32 to an attachment plate, like attachment plate 38, is possible, as long as there is an acceptable way for a patient to lock and unlock the patient interface from this anchor.

FIG. 5 shows an enlarged view of a bone anchor 24 and a perspective view wherein the post-like design can be seen in more detail.

Based on the afore-mentioned designs of the bone anchors 24 and 26, which can be used in any combination for a patient interface device 10 as described before, a patient can attach the patient interface 12 of the patient interface device 10 by connecting the coupling element 32 via a ring 36 to bone anchor 24 and/or by connecting the coupling element 34 to attachment plate 38 of the bone anchor 26.

This way the patient interface 12 is attached to the patient 14 via bone anchor 24 and/or 26 inside the patient's mouth 18. For this, the bone anchor 24 and/or 26 are permanently fixed in the patient's mouth 18 and a direct connection of the patient interface 12 to bone anchor 24 and/or 26 is achieved by coupling elements 32 and/or 36.

In order to increase the comfort level of the patient 14 the patient interface device 10 might comprise a bite plate (not shown). This bite plate might be attached to the patient interface 12.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. Patient interface device comprising:
a patient interface (12) for delivering a gas to a patient (14), and
at least one coupling element (32, 34) arranged at said patient interface (12),
wherein said patient interface (12) is attachable to the patient (14) by connecting said at least one coupling element (32, 34) to at least one tissue anchor (24, 26), said at least one tissue anchor (24, 26) being fixed to the patient (14).

2. Patient interface device of claim 1, wherein the at least one coupling element (34) comprises a solid bar.

3. Patient interface device of claim 1, wherein the at least one coupling element (32) comprises a flexible element.

4. Patient interface device of claim 1 comprising a plurality of coupling elements (32, 34).

5. Patient interface device of claim 1 comprising two or four coupling elements (32, 34).

6. Patient interface device of claim 1, wherein the at least one coupling element (32, 34) is designed to be connected to a tissue anchor (24, 26) arranged in the patient's upper jaw (28).

7. Patient interface device of claim 1, wherein the at least one coupling element (32, 34) is designed to be connected to a tissue anchor (24, 26) arranged in the patient's lower jaw (30).

8. Patient interface system comprising:
a patient interface device of anyone of claims 1 to 7 and
at least one tissue anchor (24, 26) corresponding to said at least one coupling element (32, 34) and being fixable to tissue of the patient (14).

9. Patient interface system of claim 8, wherein the at least one tissue anchor (24, 26) comprises an attachment post (25), an attachment plate (38) or a combination thereof.

10. Patient interface system of claim 8, wherein the at least one tissue anchor (24, 26) is adapted to be arranged inside the mouth (18) of the patient (14).

11. Patient interface system of claim 8, wherein the at least one tissue anchor (24, 26) is designed as a bone anchor.

12. Patient interface system of claim 11, wherein the at least one bone anchor (24, 26) is adapted to be arranged in the patient's upper jaw (28) or in the patient's lower jaw (30).

13. Patient interface system of claim 8, wherein the tissue anchor (24, 26) is fixed to the patient (14) by a screw (40), glue, cement, staples or a combination thereof.

14. Method for attaching a patient interface of a patient interface device of anyone of claims 1 to 7 to a patient, comprising:
arranging the patient interface (12) at the patient (14) and
connecting at least one coupling element (32, 34) of the patient interface system to a corresponding tissue anchor (24, 26), said tissue anchor (24, 26) being arranged to be fixed to the patient (14).

15. Use of a bone anchor (24, 26) for connecting a patient interface device of anyone of claims 1 to 7 to it.
